(19) United States
(12) Patent Application Publication
(10) Pub. No.: US 2017/0342061 A1
(43) Pub. Date: Nov. 30, 2017

(54) CRYSTAL FORM I OF CANAGLIFLOZIN AND PREPARATION METHOD THEREOF

(71) Applicant: CHONGQING PHARMACEUTICAL INDUSTRIAL RESEARCH INSTITUTE CO., LTD, Chongqing (CN)

(72) Inventors: Fei WANG, Chongqing (CN); Jian ZHANG, Chongqing (CN); Meng LIN, Chongqing (CN); Yuanfu TANG, Chongqing (CN); Hao CHEN, Chongqing (CN); Huangshu LEI, Chongqing (CN)

(73) Assignee: CHONGQING PHARMACEUTICAL INDUSTRIAL RESEARCH INSTITUTE CO., LTD, Chongqing (CN)

(21) Appl. No.: 15/538,965

(22) PCT Filed: Mar. 27, 2015
(86) PCT No.: PCT/CN2015/075180

§ 371 Date: Jun. 22, 2017

(30) Foreign Application Priority Data
Dec. 25, 2014 (CN) 201410820560.1

(51) Int. Cl. C07D 409/10

(52) U.S. Cl. CPC C07D 409/10; C07B 2200/13

(57) ABSTRACT

Disclosed in the present invention is a crystal form I of Canagliflozin, and an X-ray powder diffraction spectrum of the crystal form I has characteristic diffraction peaks when 2θ is at the position of 4.4±0.2°, 8.4±0.2°, 16.8±0.2°, 17.5±0.2°, 18.0±0.2°, and 22.8±0.2°. The crystal form is physically and chemically stable and is suitable for manufacturing of various preparations.

### FIELD

The present invention belongs to the field of medicinal chemistry, and more particularly relates to a novel crystal form of Canagliflozin which is crystal form I of Canagliflozin, and a preparation method thereof.

### BACKGROUND

Canagliflozin, which has a chemical name of 1-(β-D-glucopyranosyl)-4-methyl-3-[5-(4-fluorophenyl)-2-thienylmethyl]benzene and a structure as shown in formula I, and can be prepared by the method disclosed in CN101801371.

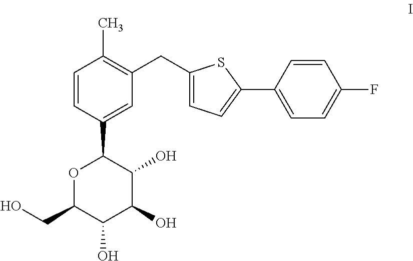 [SMILES: Cc1ccc([C@@H]2O[C@H](CO)[C@@H](O)[C@H](O)[C@H]2O)cc1Cc1ccc(-c2ccc(F)cc2)s1]

Canagliflozin is a selective type II sodium-glucose cotransporter protein (SGLT2) inhibitor which was developed by US Johnson & Johnson Company. On Mar. 29, 2013, US Food and Drug Administration (FDA) approved that it can be used in combination with diet control and exercise so as to improve glycemic control in adults with type-2 diabetes mellitus.

Canagliflozin is a poorly water soluble compound which is generally used in a solid form in formulations, and therefore, researches on its crystal form are of great significance.

CN101573368 discloses a crystal form of a hemihydrate of Canagliflozin having characteristic diffraction peaks at 2θ values of 4.36°, 13.54°, 16.00°, 19.32° and 20.80° in its X-ray powder diffraction pattern using a CuK_{α} source, which crystal form is obtained by curing in ethyl acetate/diethyl ether/water or acetone/water system.

CN101801371 discloses another crystal form having characteristic diffraction peaks at 2θ values of 10.9°, 15.5°, 17.3°, 18.8° and 20.3° in its X-ray powder diffraction pattern using a CuK_{α}, source, which crystal form is obtained by curing in ethyl acetate/n-heptane/water system.

WO2013064909 discloses co-crystals of Canagliflozin with L-proline, D-proline, L-phenylalanine, as well as an amorphous form of Canagliflozin.

CN103554092 discloses a crystal form B of Canagliflozin having characteristic diffraction peaks at 2θ values of 6.3°, 9.4° and 12.6° in its X-ray powder diffraction pattern using a CuK_{α} source, which crystal form is obtained by dissolving Canagliflozin in a mixed solvent of water and an organic solvent followed by slow evaporation at room temperature.

CN103588762 discloses a crystal form C of Canagliflozin having characteristic diffraction peaks at 2θ values of 6.5°, 9.8° and 16.4° in its X-ray powder diffraction pattern using a CuK_{α} source, which crystal form is obtained by dissolving Canagliflozin in a mixed solvent of water and an organic solvent followed by slow evaporation at room temperature. This patent further discloses a crystal form D of Canagliflozin having characteristic diffraction peaks at 2θ values of 6.8°, 13.6° and 20.5° in its X-ray powder diffraction pattern using a CuK_{α} source, which crystal form is obtained by heating the prepared crystal form C to 50˜90° C.

CN103641822 discloses another crystal form of a hemihydrate of Canagliflozin having characteristic diffraction peaks at 2θ values of 3.86°, 15.46°, 17.30°, 18.80°, 19.10° and 20.26° in its X-ray powder diffraction pattern using a CuK_{α} source, which crystal form is obtained by dissolving Canagliflozin in a good solvent, and then adding a mixed solvent of a poor solvent and water to precipitate it.

CN103980261 discloses a crystal form A of Canagliflozin having characteristic diffraction peaks at 2θ values of 3.7°, 7.7°, 7.9°, 11.5°, 13.1°, 13.5°, 14.3°, 15.5°, 17.3°, 18.8°, 19.3°, 20.3°, 22.5°, 22.7°, 23.2° and 23.4° in its X-ray powder diffraction pattern using a CuK_{α} source, which crystal form is obtained by dissolving Canagliflozin in an alcoholic solvent to formulate a suspension at 0.05˜0.5 g/ml, dissolving the suspension at 15˜43° C. and then adding 3˜10 times a solvating-out agent to precipitate it.

CN103980262 discloses another crystal form B of Canagliflozin having characteristic diffraction peaks at 2θ values of 3.4°, 6.6°, 12.6°, 13.2°, 15.3°, 15.6°, 16.5°, 19.4°, 19.8° and 23.7° in its X-ray powder diffraction pattern using a CuK_{α} source, which crystal form is obtained by dissolving Canagliflozin in an alcoholic solvent to formulate a solution at 0.1˜0.5 g/ml, and evaporating the solvent at 48˜70° C.

CN103936725 discloses another crystal form C of Canagliflozin having characteristic diffraction peaks at 2θ values of 3.4°, 6.5°, 12.7°, 15.8°, 19.8°, 24.3°, 24.8° and 29.1° in its X-ray powder diffraction pattern using a CuK_{α} source, which crystal form is obtained by dissolving Canagliflozin in an organic good solvent to formulate a solution at 0.05˜0.3 g/ml, adding a poor solvent after being dissolved, and precipitating it at −20˜10° C.

WO2014180872 discloses another crystal form of Canagliflozin having characteristic diffraction peaks at 2θ values of 5.4°, 6.7°, 13.2°, 16.1°, 19.6° and 24.1° in an X-ray powder diffraction pattern using a CuK_{α} source, which crystal form is obtained by converting an amorphous form of Canagliflozin in water.

The inventors prepared a novel crystal form of Canagliflozin during studying the crystal forms of Canagliflozin, which is prepared in a simple way, has stable physical and chemical properties, is easy to be stored and is suitable for being prepared into various preparations.

### SUMMARY

It is an object of the present invention to provide a novel crystal form of Canagliflozin, which has a simple preparation process, excellent physical and chemical stability and is suitable for manufacturing and industrial production of various preparations.

The novel crystal form of Canagliflozin provided in the present invention is defined herein as crystal form I of Canagliflozin.

The crystal form I of Canagliflozin of the present invention has characteristic diffraction peaks at positions with 2θ values of 4.4±0.2°, 8.4±0.2°, 16.8±0.2°, 17.5±0.2°, 18.0±0.2° and 22.8±0.2° in the X-ray powder diffraction pattern thereof.

The crystal form I of Canagliflozin of the present invention as described above further comprises characteristic diffraction peaks at positions with 2θ values of 12.1±0.2°, 12.6±0.2°, 15.3±0.2°, 19.3±0.2°, 20.4±0.2°, 22.2±0.2°, 23.0±0.2°, 24.6±0.2° and 26.6±0.2° in the X-ray powder diffraction pattern thereof.

In an embodiment, the crystal form I of Canagliflozin of the present invention has characteristic diffraction peaks as shown in FIG. 1 in the X-ray powder diffraction pattern thereof.

The crystal form I of Canagliflozin of the present invention has an endothermic peak between 90° C. and 95° C., particularly reaches a peak at about 93° C. in a DSC scanning graph thereof as well as has about 3.97% of weight loss when heated to 180° C. in a TGA scanning graph thereof.

The crystal form I of Canagliflozin of the present invention has characteristics as shown in FIG. 2 in the DSC-TGA scanning graph thereof.

The novel crystal form I of Canagliflozin of the present invention has a characteristic absorption peak at 1647 cm⁻¹ in the infrared absorption spectrum thereof.

In an embodiment, the novel crystal form of Canagliflozin of the present invention has characteristics as shown in FIG. 3 in the infrared absorption spectrum thereof.

The X-ray powder diffraction test on the crystal form I of Canagliflozin of the present invention was performed with a CuKα source (α=1.5406 Å) from a Shimadzu XRD-6000 X-ray diffractometer, Japan, at ambient temperature and ambient humidity. During the test, due to a variety of factors such as particle size of the test sample, treatment method of the sample when testing, instrument, test parameters, test operations and so on, there will be some differences in the peak position or peak intensity of the measured X-ray powder diffraction patterns for the same crystal form. The experimental error of the 2θ values of the diffraction peaks in X-ray powder diffraction patterns may be within ±0.2°. “Ambient temperature” is typically 0˜40° C. and “ambient humidity” is typically 30%˜80% of relative humidity.

The DSC-TGA analysis of the crystal form I of Canagliflozin of the present invention was performed at ambient temperature and ambient humidity by using a Mettler 1100LF type instrument, Switzerland. The test was carried out by purging with a high-purity Ar gas at a flow rate of 50 ml/min and programmed warming at a rate of 10° C./min, with the range of temperature rise being from room temperature to 300° C. “Ambient temperature” is typically 0˜40° C. and “ambient humidity” is typically 30%˜80% of relative humidity.

The IR spectrum analysis of the crystal form I of Canagliflozin of the present invention was tested by Fourier Transform Infrared Spectrometer (Nicolet Atavar FT-IR330) from Nicolet company at a relative humidity of typically less than 80% and a temperature of typically 15˜30° C. During the test, a tablet was pressed with KBr, and the spectrophotometer was calibrated with polystyrene (wavelength). During the test, due to a variety of factors (such as the size of the ground particles, the degree of tabletting, and the relative humidity in the air and so on), there will be some differences in the peak position or peak intensity of the measured IR spectra. The experimental error of the characteristic absorption peak values in the IR spectra may be within ±2 cm⁻¹.

An object of the present invention is to further provide a method for preparing crystal form I of Canagliflozin, which comprises heating and dissolving Canagliflozin in a mixed solvent of a good solvent and water, and then adding water to precipitate Canagliflozin.

In a specific embodiment, the method for preparing crystal form I of Canagliflozin of the present invention comprises the following steps:

1) dissolving Canagliflozin with a mixed solvent of a suitable good solvent and water to obtain a Canagliflozin solution, wherein the dissolving temperature is 30˜100° C., preferably 50˜80° C.;

2) then reducing the temperature of the Canagliflozin solution to 20˜60° C., preferably 30˜50° C., and adding water to precipitate Canagliflozin;

3) separating the precipitated solid by filtration or centrifugation;

4) optionally, drying the separated solid at a drying temperature of generally 30˜80° C., preferably 40˜50° C., wherein the drying can be drying under an ambient pressure, or drying under a reduced pressure with the vacuum degree being typically 300˜760 mmHg, preferably 650˜760 mmHg.

In the specific embodiment as described above, in the method of the present invention, the suitable good solvent in step 1) includes methanol, ethanol, isopropanol, acetone, tetrahydrofuran, N,N-dimethyl formamide, dimethyl sulfoxide, N,N-dimethyl acetamide, dioxane, or a mixture thereof, preferably methanol, ethanol, isopropanol or a mixture thereof.

In the specific embodiment as described above, in the method of the present invention, the volume ratio of the good solvent to water is 1:1˜3. The weight-to-volume ratio of Canagliflozin to the good solvent is 1:3˜8 g/ml.

In a preferred specific embodiment, the method for preparing crystal form I of Canagliflozin of the present invention comprises the following steps:

1) dissolving Canagliflozin with a mixed solvent of a suitable good solvent and water to obtain a Canagliflozin solution, wherein a dissolving temperature is 50˜80° C., and the suitable good solvent is selected from methanol, ethanol, and isopropanol;

2) then reducing the temperature of the Canagliflozin solution to 30˜50° C., and adding water to precipitate Canagliflozin;

3) separating the precipitated solid by filtration or centrifugation;

4) optionally, drying the separated solid under a reduced pressure, wherein the drying temperature is 30˜80° C., and the vacuum degree is 650˜760 mmHg, wherein the volume ratio of the suitable good solvent to water is 1:1˜3.

In the preferred specific embodiments as described above, the weight-to-volume ratio of Canagliflozin to the good solvent is 1:3˜8 g/ml.

In the embodiments as described above, in the method of the present invention, the precipitation of Canagliflozin is generally completed under a stirring condition.

To illustrate the stability of the crystal form I of Canagliflozin of the present invention, the crystal form I of Canagliflozin prepared in Example 1 was selected for stability studies, and the results are shown in the table below.

TABLE
Stability testing results of crystal form I of Canagliflozin

| | HPLC change | | whether the | whether there | |
|---|---|---|---|---|---|
| | before | after | appearance | being apparent | |
| test conditions | placed | placed | changed | moisture absorption | crystal form |
| placed at a temperature of | 99.89% | 99.89% | No change | No apparent | Crystal form I |
| 25 ± 2° C. and a humidity of | | | | moisture absorption | |
| RH92.5% for 30 days | | | | | |
| Placed at a temperature of | 99.89% | 99.88% | No change | No apparent | Crystal form I |
| 40 ± 2° C. for 30 days | | | | moisture absorption | |
| placed at a temperature of | 99.89% | 99.87% | No change | No apparent | Crystal form I |
| 60 ± 2° C. for 30 days | | | | moisture absorption | |

As can be seen from the above table, the crystal form I of Canagliflozin of the present invention has a good stability and is favourable to be prepared into various preparations.

The crystal form I of Canagliflozin of the present invention has a simple preparation process, which can be completed by using common equipments and mild conditions, and is suitable for industrialized production.

### BRIEF DESCRIPTION OF THE DRAWINGS

FIG. 1 is an X-ray diffraction pattern of the crystal form I of Canagliflozin of the present invention.

FIG. 2 is a DSC-TGA graph of the crystal form I of Canagliflozin of the present invention.

FIG. 3 is an infrared spectrum of the crystal form I of Canagliflozin of the present invention.

### DETAILED DESCRIPTION OF EMBODIMENTS

Hereinafter, the present invention will be further illustrated in conjunction with examples, which allow those skilled in the art to understand the substance of the present invention more completely and are not intended to limit the scope of the invention in any way.

The X-ray powder diffraction patterns as described in the present invention were collected on a Shimadzu XRD-6000 X-ray diffractometer, Japan. The parameters of the X-ray powder diffraction analysis of the present invention were as follows:

X-ray reflection parameter: CuKα

CuKα source (α=1.5406 Å)

Voltage: 40 kilovolts (KV)

Current: 30 milliamperes (mA)

Divergence slit: automatic

Scanning mode: continuous

Scanning range: 2˜45 degrees

Sampling width: 0.02 degree

Scanning speed: 2 degree/minute

The DSC-TGA graphs of the crystal form I of Canagliflozin of the present invention were collected on a Mettler 1100LF type instrument, Switzerland. The parameters of the DSC-TGA analysis of the present invention were as follows:

Temperature range: room temperature˜300° C.

Scanning rate: 10° C./min

Protection gas: Ar gas 50 ml/min

The IR spectra (KBr tablet) of the crystal form I of Canagliflozin of the present invention were collected on a Fourier Transform Infrared spectrometer (Nicolet Atavar FT-IR330) from US Nicolet company.

### Example 1

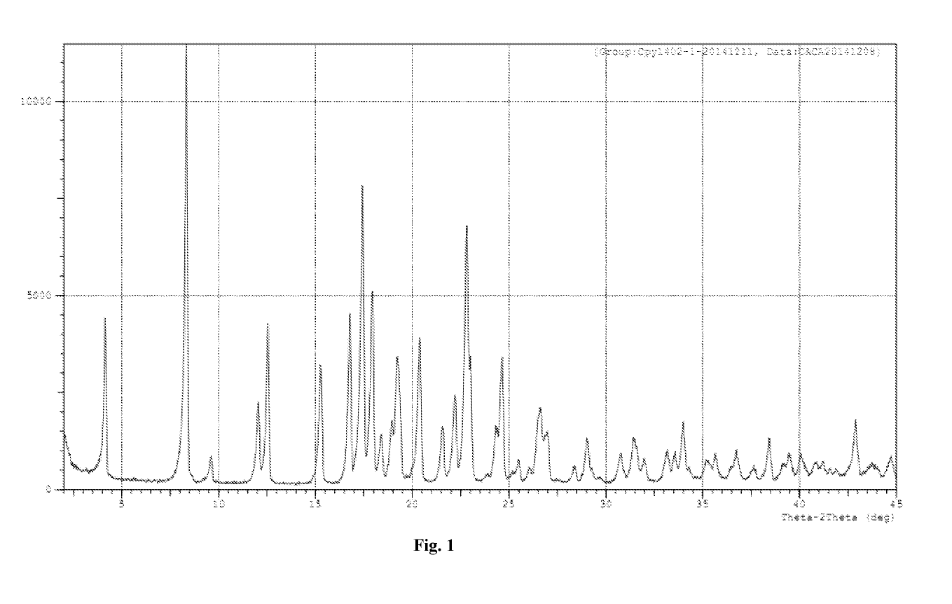

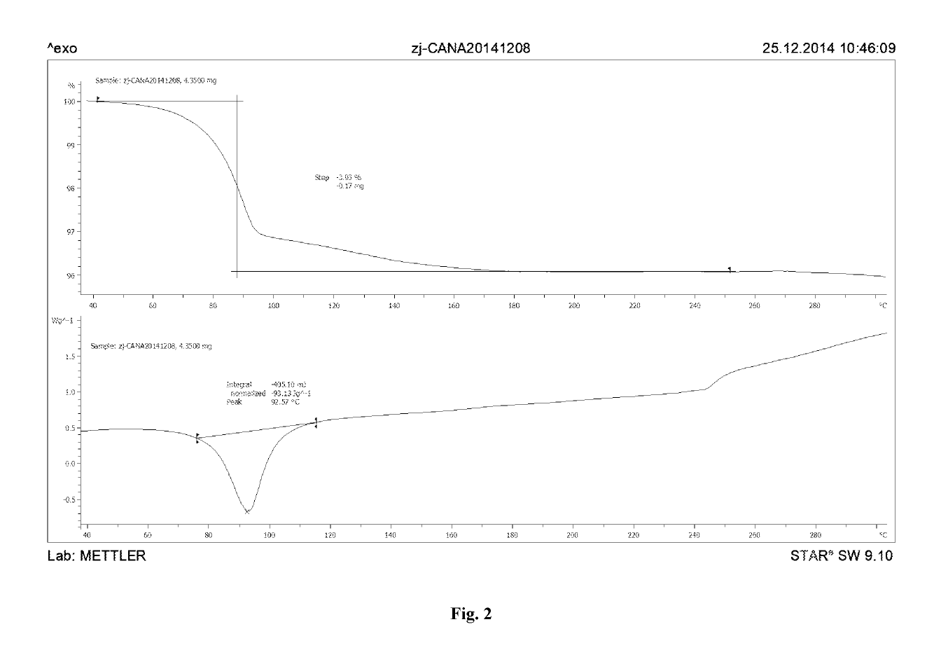

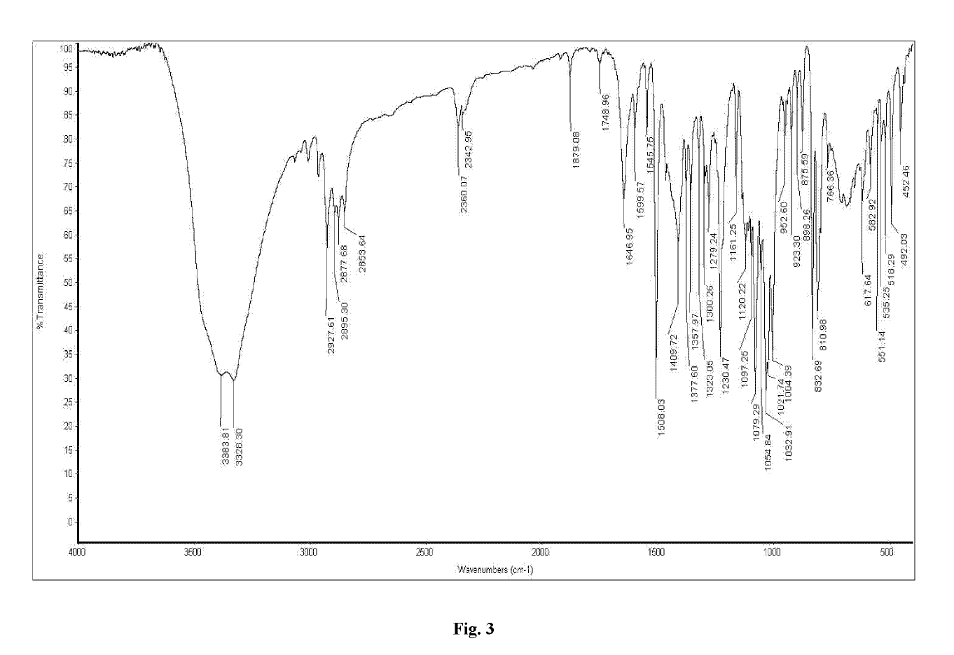

100 g Canagliflozin was dissolved in a mixed solvent of 300 ml methanol and 100 ml water at 50˜55° C., cooled to a temperature of 30˜35° C., and 200 ml water was added dropwise under stirring. After the completion of the addition, a mass of solid precipitated out and was filtered. The filter cake was dried under a reduced pressure at 700˜760 mmHg and 40˜50° C. to obtain 96 g Canagliflozin, HPLC: 99.89%. The test result for X-ray powder diffraction is shown in FIG. 1; the test result for DSC-TGA is shown in FIG. 2; and the test result for infrared spectrum is shown in FIG. 3.

### Example 2 Preparation of Crystal Form I of Canagliflozin

80 g Canagliflozin was dissolved in a mixed solvent of 350 ml ethanol and 150 ml water at 65˜70° C., cooled to a temperature of 35˜40° C., and 550 ml water was added dropwise under stirring. After the completion of the addition, a mass of solid precipitated out, cooled to room temperature, and filtered. The filter cake was dried under a reduced pressure at 650˜760 mmHg and 45˜50° C. to obtain 67 g Canagliflozin, HPLC: 99.88%. It was confirmed by X-ray powder diffraction analysis as crystal form I of Canagliflozin.

### Example 3 Preparation of Crystal Form I of Canagliflozin

100 g Canagliflozin was dissolved in a mixed solvent of 800 ml isopropanol and 300 ml water at 70˜80° C., cooled to a temperature of 40˜50° C., and 2100 ml water was added dropwise under stirring. After the completion of the addition, a mass of solid precipitated out, cooled to room temperature and filtered. The filter cake was dried under a reduced pressure at 680˜760 mmHg and 45˜50° C. to obtain 92 g Canagliflozin, HPLC: 99.92%. It was confirmed by X-ray powder diffraction analysis as a crystal form I of Canagliflozin.

1. A crystal form I of Canagliflozin, having characteristic diffraction peaks at positions with 2θ values of 4.4±0.2°, 8.4±0.2°, 16.8±0.2°, 17.5±0.2°, 18.0±0.2°, and 22.8±0.2° in an X-ray powder diffraction pattern thereof.

2. The crystal form I of claim 1, further comprising characteristic diffraction peaks at positions with 2θ values of 12.1±0.2°, 12.6±0.2°, 15.3±0.2°, 19.3±0.2°, 20.4±0.2°, 22.2±0.2°, 23.0±0.2°, 24.6±0.2°, and 26.6±0.2° in the X-ray powder diffraction pattern thereof.

3. The crystal form I of claim 1, substantially having characteristic diffraction peaks as shown in FIG. 1 in the X-ray powder diffraction pattern thereof.

4. The crystal form I of claim 1, having an endothermic peak between 90° C. and 95° C. in a DSC scanning graph thereof.

5. A method for preparing the crystal form I of Canagliflozin of claim 1, comprising heating and dissolving Canagliflozin in a mixed solvent of a suitable good solvent and water, then adding water to precipitate Canagliflozin.

6. The method of claim 5, specifically comprising the following steps:
1) dissolving Canagliflozin with a mixed solvent of a suitable good solvent and water to obtain a Canagliflozin solution, wherein the dissolving temperature is 30˜100° C.;
2) reducing the temperature of the Canagliflozin solution to 20˜60° C., and adding water to precipitate Canagliflozin;
3) separating the precipitated solid by filtration or centrifugation;
4) optionally, drying the separated solid at atmospheric or reduced pressure conditions, wherein the drying temperature is 30˜80° C.

7. The method of claim 6, wherein the dissolving temperature in step 1) is 50˜80° C., the temperature in step 2) is 30˜50° C., and the drying temperature in step 4) is 40˜50° C.

8. The method of claim 5, wherein the suitable good solvent includes methanol, ethanol, isopropanol, acetone, tetrahydrofuran, N,N-dimethyl formamide, dimethyl sulfoxide, N,N-dimethyl acetamide, dioxane, or a mixture thereof.

9. The method of claim 8, wherein the suitable good solvent is methanol, ethanol, isopropanol or a mixture thereof.

10. The method of claim 5, wherein the volume ratio of the suitable good solvent to water is 1:1-3.
